(19) United States
(12) Patent Application Publication
(10) Pub. No.: US 2024/0361405 A1
(43) Pub. Date: Oct. 31, 2024

(54) DEPLOYABLE GUARD FOR PORTABLE MAGNETIC RESONANCE IMAGING DEVICES

(71) Applicant: Hyperfine Operations, Inc., Guilford, CT (US)

(72) Inventors: Richard Q. Wang, New Haven, CT (US); Jonathan C. Schultz, Guilford, CT (US); Christopher Thomas McNulty, Guilford, CT (US); Ethan Liu, New Haven, CT (US); Yin Shing Chong, Middletown, CT (US); Timothy Bernhardt, Cheshire, CT (US)

(73) Assignee: Hyperfine Operations, Inc., Guilford, CT (US)

(21) Appl. No.: 18/769,295

(22) Filed: Jul. 10, 2024

Related U.S. Application Data
(63) Continuation of application No. PCT/US2023/010558, filed on Jan. 11, 2023.
(60) Provisional application No. 63/298,542, filed on Jan. 11, 2022.

(51) Int. Cl. G01R 33/28

(52) U.S. Cl. CPC G01R 33/288

(57) ABSTRACT

Some aspects of the technology described herein provide for a deployable guard device configured to be coupled to a portable magnetic resonance imaging (MRI) device, the deployable guard device comprising: a plurality of arms that, when the deployable guard device is in a deployed position, at least partially surround a region within which a magnetic field strength of a magnetic field generated by the portable MRI device equals or exceeds a threshold field strength; and at least one deployment device configured to generate a force to facilitate moving the deployable guard device from an undeployed position to the deployed position.

### CROSS-REFERENCE TO RELATED APPLICATIONS

This application claims the benefit under 35 U.S.C. § 120 as a continuation of PCT Patent Application No. PCT/US2023/010558, filed on Jan. 11, 2023, which claims benefit under 35 U.S.C. § 119 (e) to U.S. provisional patent application Ser. No. 63/298,542, filed on Jan. 11, 2022, and titled “DEPLOYABLE GUARD FOR PORTABLE MAGNETIC RESONANCE IMAGING DEVICES,” the disclosures of which are incorporated herein by reference in their entirety.

### BACKGROUND

MRI provides an important imaging modality for numerous applications and is widely utilized in clinical and research settings to produce images of the inside of the human body. As a generality, MRI is based on detecting magnetic resonance (MR) signals, which are electromagnetic waves emitted by atoms in response to state changes resulting from applied electromagnetic fields. For example, nuclear magnetic resonance (NMR) techniques involve detecting MR signals emitted from the nuclei of excited atoms upon the re-alignment or relaxation of the nuclear spin of atoms in an object being imaged (e.g., atoms in the tissue of the human body). Detected MR signals may be processed to produce images, which in the context of medical applications, allows for the investigation of internal structures and/or biological processes within the body for diagnostic, therapeutic and/or research purposes.

MRI provides an attractive imaging modality for biological imaging due to the ability to produce non-invasive images having relatively high resolution and contrast without the safety concerns of other modalities (e.g., without needing to expose the subject to ionizing radiation, e.g., x-rays, or introducing radioactive material to the body). Additionally, MRI is particularly well suited to provide soft tissue contrast, which can be exploited to image subject matter that other imaging modalities are incapable of satisfactorily imaging. Moreover, MR techniques are capable of capturing information about structures and/or biological processes that other modalities are incapable of acquiring. However, there are a number of drawbacks to MRI that, for a given imaging application, may involve the relatively high cost of the equipment, limited availability (e.g., difficulty in gaining access to clinical MRI scanners) and/or the length of the image acquisition process.

The trend in clinical MRI has been to increase the field strength of MRI scanners to improve one or more of scan time, image resolution, and image contrast, which, in turn, continues to drive up costs. The vast majority of installed MRI scanners operate at 1.5 or 3 tesla (T), which refers to the field strength of the main magnetic field B₀. A rough cost estimate for a clinical MRI scanner is approximately one million dollars per tesla, which does not factor in the substantial operation, service, and maintenance costs involved in operating such MRI scanners.

These high-field MRI devices typically require large superconducting magnets and associated electronics to generate a strong uniform static magnetic field (B₀) in which an object (e.g., a patient) is imaged. The size of such systems is considerable with a typical high-field MRI installment including multiple rooms for the magnet, electronics, thermal management system, and control console areas. The size and expense of high-field MRI devices generally limits their usage to facilities, such as hospitals and academic research centers, which have sufficient space and resources to purchase and maintain them. The high cost and substantial space requirements of high-field MRI devices results in limited availability of MRI scanners. As such, there are frequently clinical situations in which an MRI scan would be beneficial, but due to one or more of the limitations discussed above, is not practical or is impossible.

A further consideration related to MRI devices of any field strength are stray magnetic fields produced outside the imaging region of the MRI devices (also known as fringe fields), which are measured in Gauss. Depending on their strength, fringe fields may be dangerous to bystanders and may interfere with nearby electronics including medical devices (e.g., pacemakers) and computers (e.g., smartphones).

### SUMMARY

Some embodiments provide for a deployable guard device configured to be coupled to a portable magnetic resonance imaging (MRI) device, the deployable guard device comprising: a plurality of arms that, when the deployable guard device is in a deployed position, at least partially surround a region within which a magnetic field strength of a magnetic field generated by the portable MRI device equals or exceeds a threshold field strength; and at least one deployment device configured to generate a force to facilitate moving the deployable guard device from an undeployed position to the deployed position.

Some embodiments provide for a deployable guard device configured to be coupled to a portable magnetic resonance imaging (MRI) device, the deployable guard device comprising: a plurality of arms that, when the deployable guard device is in a deployed position, at least partially surround a region within which a magnetic field strength of a magnetic field generated by the portable MRI device equals or exceeds a threshold field strength; and at least one deployment means for moving the deployable guard device from an undeployed position to the deployed position

Some embodiments provide for a system, comprising: a portable magnetic resonance imaging (MRI) device; and a deployable guard device coupled to the portable MRI device, the deployable guard device comprising: a plurality of arms that, when the deployable guard device is in a deployed position, at least partially surround a region within which a magnetic field strength of a magnetic field generated by the portable MRI device equals or exceeds a threshold field strength; and at least one deployment device configured to generate a force to facilitate moving the deployable guard device from an undeployed position to the deployed position.

Some embodiments provide for a method for operating a magnetic resonance imaging (MRI) device, the MRI device being coupled to a deployable guard device comprising a plurality of arms that, when the deployable guard device is in a deployed position, at least partially surround a region within which a magnetic field strength of a magnetic field generated by the MRI device equals or exceeds a threshold, the method comprising: moving the deployable guard device from an undeployed position to the deployed position, wherein: the deployable guard device further comprises at least one deployment device configured to generate a first force to facilitate moving the deployable guard device from the undeployed position to the deployed position, subsequent to moving the deployable guard device to the deployed position, transporting the MRI device from a first location to a second location; and moving the deployable guard device from the deployed position to the undeployed position.

Some embodiments provide for a deployable guard device configured to be coupled to a portable magnetic resonance imaging (MRI) device, the deployable guard device comprising: a support for coupling to the portable MRI device; and a plurality of arms coupled to the support and that, when the deployable guard device is in a deployed position, at least partially surround a region within which a magnetic field strength of a magnetic field generated by the portable MRI device equals or exceeds a threshold field strength, the plurality of arms including a first arm, wherein applying a force to the first arm to move the first arm from its undeployed position toward its deployed position causes the support to move one or more other arms of the plurality of arms from their respective undeployed positions toward their respective deployed positions.

Some embodiments provide for a system, comprising: a portable magnetic resonance imaging (MRI) device; and a deployable guard device coupled to the portable MRI device, the deployable guard device comprising: a support coupled to the portable MRI device; and a plurality of arms that, when the deployable guard device is in a deployed position, at least partially surround a region within which a magnetic field strength of a magnetic field generated by the portable MRI device equals or exceeds a threshold; and wherein applying a force to the first arm to move the first arm from its undeployed position toward its deployed position causes the support to move one or more other arms of the plurality of arms from their respective undeployed positions toward their respective deployed positions.

Some embodiments provide for a method for operating a magnetic resonance imaging (MRI) device, the MRI device being coupled to a deployable guard device comprising a support coupled to the MRI device and a plurality of arms coupled to the support and that, when the deployable guard device is in a deployed position, at least partially surround a region within which a magnetic field strength of a magnetic field generated by the MRI device equals or exceeds a threshold, the method comprising: moving the deployable guard device from the deployed position to an undeployed position, wherein the deployable guard device is configured to move between the deployed position and the undeployed position in response to a force on only one of the plurality of arms; imaging, using the MRI device; and subsequent to imaging, moving the deployable guard device from the undeployed position to the deployed position.

### BRIEF DESCRIPTION OF THE DRAWINGS

Various aspects and embodiments of the application will be described with reference to the following figures. It should be appreciated that the figures are not necessarily drawn to scale. Items appearing in multiple figures are indicated by the same reference number in all the figures in which they appear.

FIG. 1 illustrates an example magnetic resonance imaging device for use in accordance with some embodiments of the technology described herein.

FIGS. 2A, 2B and 2C are top, front and side views of the example magnetic resonance imaging device of FIG. 1, illustrating example magnetic fringe fields associated with the device, in accordance with some embodiments of the technology described herein.

FIG. 2D illustrates magnetic field strength of a fringe field for another example magnetic resonance imaging device, in accordance with some embodiments of the technology described herein.

FIG. 3A illustrates an example deployable guard device in a deployed position, in accordance with some embodiments of the technology described herein.

FIG. 3B illustrates the example deployable guard device of FIG. 3A coupled to a portable magnetic resonance imaging device and in an undeployed position, in accordance with some embodiments of the technology described herein.

FIG. 3C illustrates the example deployable guard device of FIG. 3A coupled to a portable magnetic resonance imaging device and in an undeployed position, in accordance with some embodiments of the technology described herein.

FIG. 4A illustrates another configuration of the deployable guard device in a deployed position, in accordance with some embodiments of the technology described herein.

FIG. 4B illustrates the example deployable guard device of FIG. 4A in an undeployed position, in accordance with some embodiments of the technology described herein.

FIG. 5A illustrates the example deployable guard device of FIG. 4A coupled to a portable magnetic resonance imaging device and in the deployed position, in accordance with some embodiments of the technology described herein.

FIG. 5B illustrates the example deployable guard device of FIG. 4A coupled to a portable magnetic resonance imaging device and in the undeployed position, in accordance with some embodiments of the technology described herein.

FIG. 5C illustrates the example deployable guard device of FIG. 4A coupled to a portable magnetic resonance imaging device and in the undeployed position, in accordance with some embodiments of the technology described herein.

FIG. 6A illustrates another example deployable guard device in a deployed position, in accordance with some embodiments of the technology described herein.

FIG. 6B illustrates the deployable guard device of FIG. 6A in a position between the deployed position and an undeployed position, in accordance with some embodiments of the technology described herein.

FIG. 6C illustrates the deployable guard device of FIG. 6A in the undeployed position, in accordance with some embodiments of the technology described herein.

FIG. 7A illustrates the deployable guard device of FIG. 6A coupled to a portable magnetic resonance imaging device and in the deployed position, in accordance with some embodiments of the technology described herein.

FIG. 7B illustrates the deployable guard device of FIG. 6A coupled to a portable magnetic resonance imaging device and in the undeployed position, in accordance with some embodiments of the technology described herein.

FIG. 7C illustrates the deployable guard device of FIG. 6A coupled to a portable magnetic resonance imaging device and in the undeployed position, in accordance with some embodiments of the technology described herein.

FIG. 8A illustrates a support of the deployable guard device of FIG. 6A, in accordance with some embodiments of the technology described herein.

FIG. 8B illustrates aspects of coupling the deployable guard device of FIG. 6A to the support of FIG. 8A, in accordance with some embodiments of the technology described herein.

FIGS. 9A-9C illustrate motion of the deployable guard device of FIG. 6A when the deployable guard device is moved from the deployed position to the undeployed position, in accordance with some embodiments of the technology described herein.

FIG. 10 illustrates a deployment device for automatically moving a deployable guard device from an undeployed position to a deployed position, in accordance with some embodiments of the technology described herein.

FIG. 11 illustrates a method for imaging using a magnetic resonance imaging device coupled to a deployable guard device, in accordance with some embodiments of the technology described herein.

### DETAILED DESCRIPTION

The present disclosure relates generally to magnetic resonance imaging (MRI) devices and, more specifically, a deployable guard suitable for use with portable MRI devices. Aspects of the deployable guard device described herein allow for easier transitioning between deployed and undeployed positions of the deployable guard device.

Recently, portable low-field MRI devices have been developed which can be implemented as self-contained systems that are deployable in a wide variety of clinical settings where high-field MRI devices cannot, for example, by virtue of being transportable, cartable or otherwise generally mobile so as to be deployable where needed. As a result of this portability, such low-field MRI devices may be expected to operate in generally unshielded or partially shielded environments.

With the emergence of a new paradigm for MRI, certain additional challenges may arise with respect to a portable, point-of-care (POC) MRI device that can be installed in a variety of settings such as an emergency room, office or clinic. For example, when in storage or when transported from location to location, a portable, low-field POC MRI device (including any of the systems described herein) may temporarily reside in (or pass through) an area or areas that are not access controlled. On the one hand, a low-field system MRI device operates at a static magnetic field much lower than that of conventional high-field MRI devices, and as such certain risks typically associated with high-field systems (e.g., potential projectile effects) are likely absent. On the other hand, there still may be other concerns associated with having even low-level static magnetic fields present in areas that are not access controlled. Examples of such concerns may include, but are not necessarily limited to: individuals having active implants (e.g., pacemakers, defibrillators, insulin pumps, deep brain stimulators, vagus nerve stimulators, cochlear implants, etc.) in the vicinity of the MRI device; individuals with metal containing tattoos or permanent make-up on the head or neck regions in the vicinity of the MRI device; and individuals with suspected metal present in the eye (e.g., metal workers, injury victim, etc.) in the vicinity of the MRI device.

High fringe fields (e.g., magnetic fields extending beyond an imaging region of an MRI device) may be dangerous to bystanders for the reasons discussed herein, however low-strength fringe fields (e.g., fringe fields having a strength of less than 30 Gauss, less than 25 Gauss, less than 20 Gauss, less than 15 Gauss, less than 10 Gauss, less than 5 Gauss, less than 2 Gauss, less than 1 Gauss, any strength in the range of 2-10 Gauss, 1-30 Gauss, 5-20 Gauss, or 2-20 Gauss, etc.) may be tolerated because such low-strength fringe fields may not present a safety concern or otherwise interfere with operation of nearby electronics including implants (e.g., pacemakers) or other electronic devices (e.g., medical instruments, smartphones, etc.).

In some environments, safety regulations require indications of the boundary or perimeter within which the magnetic field of the MRI device exceeds a given threshold field strength. These boundaries are sometimes called “Gauss lines.” A Gauss line for a device may indicate a region, outside of which, the strength of a magnetic field generated by the device is less than a threshold strength. For example, the 5 Gauss line for an MRI device may indicate a region outside of which the magnetic field generated by the MRI device has a strength of less than 5 Gauss. Magnetic fields having strength higher than 30 Gauss may present projectile hazards. Some safety regulations require the 5, 10 and 200 Gauss lines to be indicated to demarcate the physical perimeters within which the respective thresholds are exceeded.

It should be appreciated that such challenges are generally not of concern with respect to the more conventional, high field MRI devices that are typically immobile and installed in specialized rooms with extensive shielding and defined access control protocols. For example, compliance with the above-mentioned safety regulations may be achieved by indicating the 5, 10 and 200 Gauss lines on the floor of the room in which the MRI device is installed, to remind personnel where the respective protocols need to be enforced. This solution is generally inapplicable in the context of portable MRI devices because the MRI device is moved from place to place and reapplying floor markings each time the MRI device is moved is impractical.

In view of this and as described herein, embodiments of the disclosure provide for a deployable guard device, configured to be coupled to a portable medical imaging device. When deployed, the deployable guard device is configured to inhibit encroachment within a physical boundary with respect to the portable medical imaging device. For example, in some embodiments, the deployable guard device, when in a deployed position, may provide a physical barrier to encroachment such that the region within the physical barrier includes a particular Gauss line (e.g., the 5 Gauss line, the 10 Gauss line, etc.). To this end, the deployable guard device may be configured such that, when deployed, the outer perimeter of the deployable guard device extends beyond the particular Gauss line relative to the portable MRI device to which the deployable guard device is coupled.

The inventors have developed a deployable guard device having features which facilitate transitioning the deployable guard device between its deployed and undeployed positions. As described herein, the deployable guard device may be in a deployed position to inhibit encroachment within a physical boundary of a portable medical imaging device (such as an MRI device). The deployable guard device may be temporarily in an undeployed position, for example, when in storage, when moving through doorways or confined spaces (e.g., through a narrow hallway), or when in operation. Subsequently, the deployable guard device may be moved to the deployed position, for example, when moving the deployable guard device from one location to another, through unmarked and/or unshielded areas. Accordingly, the inventors have recognized that a deployable guard device which easily transitions between the deployable and undeployable positions is advantageous to ensure that the deployable guard device is in the deployed position when necessary.

Some aspects of the deployable guard device include a deployment device which facilitates moving the deployable guard device from the undeployed position to the deployed position. For example, the deployment device may generate a force on the deployable guard device which causes the deployable guard device to automatically move to the deployed position (e.g., after temporarily moving to the undeployed position when moving through a doorway or other confined space). Accordingly, there is less need for operator intervention to ensure the deployable guard device returns to the deployed position. A deployment device which facilitates automatically moving the deployable guard device from an undeployed position to a deployed position and/or from a deployed position to an undeployed position may be implemented for use with any of the deployable guard device designs described herein.

As described herein, the deployable guard device may comprise multiple components, including multiple arms, which form a boundary. In some embodiments, the deployable guard device may be configured to move between the undeployed and deployed positions in response to a force on only some of the deployable guard device components (e.g., on only some, including only one, of the deployable guard device arms). Accordingly, transitioning between undeployed and deployed positions requires less interaction from an operators. The inventors have recognized that an asymmetrical guard device may be beneficial in circumstances where the magnetic field generated by the MRI device to which the asymmetrical guard device is coupled, as the guard device does not occupy more space than necessary (e.g., as opposed to a symmetrical guard device which may overcompensate the region within which it is necessary to inhibit encroachment). Accordingly, the asymmetrical guard device allows the portable MRI device to be moved through (e.g., transported) or stored in confined spaces, such as through a doorway or closer to a wall.

Accordingly, according to some embodiments there is provided a deployable guard device configured to be coupled to a portable magnetic resonance imaging (MRI) device, the deployable guard device comprising: a plurality of arms (e.g., at least four arms) that, when the deployable guard device is in a deployed position, at least partially surround a region within which a magnetic field strength of a magnetic field generated by the portable MRI device equals or exceeds a threshold field strength; and at least one deployment device (e.g., a torsion spring) configured to generate a force to facilitate moving the deployable guard device from an undeployed position to the deployed position.

In some embodiments, the at least one deployment device comprises a respective deployment device coupled to each of the plurality of arms.

In some embodiments, the deployable guard device is radially asymmetrical. In some embodiments, the deployable guard device is bilaterally asymmetrical.

In some embodiments, at least some arms of the plurality of arms are movable to the deployed position while others of the plurality of arms remain in an undeployed position.

In some embodiments, the plurality of arms comprises a first arm and a second arm; and the first and second arms at least partially overlap one another in space when the deployable guard device is in the deployed position. In some embodiments, the first and second arms are not coupled together at a point where the first and second arms at least partially overlap each other. In some embodiments, a first end of the first arm overlaps a first end of the second arm when the deployable guard device is in the deployed position.

In some embodiments, at least two arms of the plurality of arms are configured to move from the deployed position to the undeployed position in response to a force on only one of the at least two arms the plurality of arms. In some embodiments, at least two arms of the plurality of arms are configured to move from the undeployed position to the deployed position in response to a force on only one of the at least two of the plurality of arms.

In some embodiments, the at least one deployment device is configured to automatically return the deployable guard device to the deployed position in response to a force on the deployable guard device towards the undeployed position.

According to some embodiments there is provided a deployable guard device configured to be coupled to a portable magnetic resonance imaging (MRI) device, the deployable guard device comprising: a plurality of arms (e.g., at least four arms) that, when the deployable guard device is in a deployed position, at least partially surround a region within which a magnetic field strength of a magnetic field generated by the portable MRI device equals or exceeds a threshold field strength; and at least one deployment means for moving the deployable guard device from an undeployed position to the deployed position.

In some embodiments, the at least one deployment means comprises a respective deployment means for each of the plurality of arms.

In some embodiments, the deployable guard device is radially asymmetrical. In some embodiments, the deployable guard device is bilaterally asymmetrical.

In some embodiments, at least some arms of the plurality of arms are movable to the deployed position while others of the plurality of arms remain in an undeployed position.

In some embodiments, the plurality of arms comprise a first arm and a second arm and the first and second arms at least partially overlaps one another in space when the deployable guard device is in the deployed position. In some embodiments, the first and second arms are not coupled together at a point where the first and second arms at least partially overlap each other. In some embodiments, a first end of the first arm overlaps a first end of the second arm when the deployable guard device is in the deployed position.

In some embodiments, at least two arms of the plurality of arms are configured to move from the deployed position to the undeployed position in response to a force on only one of the at least two arms the plurality of arms. In some embodiments, at least two arms of the plurality of arms are configured to move from the undeployed position to the deployed position in response to a force on only one of the at least two of the plurality of arms.

According to some aspects, there is provided a system, comprising: a portable magnetic resonance imaging (MRI) device; and a deployable guard device coupled to the portable MRI device, the deployable guard device comprising: a plurality of arms that, when the deployable guard device is in a deployed position, at least partially surround a region within which a magnetic field strength (e.g., within a range from about 5 Gauss to about 70 Gauss, within of a magnetic field generated by the portable MRI device equals or exceeds a threshold field strength, within a range from about 1 Gauss to about 70 Gauss); and at least one deployment device configured to generate a force to facilitate moving the deployable guard device from an undeployed position to the deployed position. In some embodiments, the deployable guard device is coupled to the portable MRI device above an imaging region of the portable MRI device.

According to some aspects, there is provided a method for operating a magnetic resonance imaging (MRI) device, the MRI device being coupled to a deployable guard device comprising a plurality of arms that, when the deployable guard device is in a deployed position, at least partially surround a region within which a magnetic field strength of a magnetic field generated by the MRI device equals or exceeds a threshold, the method comprising: moving the deployable guard device from an undeployed position to the deployed position, wherein: the deployable guard device further comprises at least one deployment device configured to generate a first force to facilitate moving the deployable guard device from the undeployed position to the deployed position; subsequent to moving the deployable guard device to the deployed position, transporting the MRI device from a first location to a second location; and moving the deployable guard device from the deployed position to the undeployed position.

In some embodiments, moving the deployable guard device from the undeployed position to the deployed position comprises applying a second force on only one of the plurality of arms. In some embodiments, the method further comprises, subsequent to moving the deployable guard device to the undeployed position, imaging, using the MRI device.

According to some aspects, there is provided a deployable guard device configured to be coupled to a portable magnetic resonance imaging (MRI) device, the deployable guard device comprising: a support for coupling to the portable MRI device; and a plurality of arms (e.g., at least 8 arms) coupled to the support and that, when the deployable guard device is in a deployed position, at least partially surround a region within which a magnetic field strength of a magnetic field generated by the portable MRI device equals or exceeds a threshold field strength, the plurality of arms including a first arm, wherein applying a force to the first arm to move the first arm from its undeployed position toward its deployed position causes the support to move one or more other arms of the plurality of arms from their respective undeployed positions toward their respective deployed positions.

In some embodiments, applying a second force to the first arm to move the first arm from its deployed position toward its undeployed position causes the support to move the one or more other arms of the plurality of arms from their respective deployed positions toward their respective undeployed positions.

In some embodiments, first ends of each of the plurality of arms slide along the support when the deployable guard device moves between the deployed position and the undeployed position.

In some embodiments, the support is rotatable. In some embodiments, the support rotates along a track coupled to the portable MRI device. In some embodiments, the first arm of the plurality of arms comprises plastic. In some embodiments, the first arm of the plurality of arms comprises a fixed bend between endpoints of the first arm. In some embodiments, the first arm of the plurality of arms has a color value of at least 3.

In some embodiments, a second end of the first arm overlaps a second arm of the plurality of arms when the deployable guard device is in the deployed position. In some embodiments, the second end of the first arm is not coupled to the second arm. In some embodiments, the second end of the first arm overlaps a point on the second arm between first and second ends of the second arm when the deployable guard device is in the deployed position.

According to some embodiments, there is provided a system comprising: a portable magnetic resonance imaging (MRI) device; and a deployable guard device coupled to the portable MRI device, the deployable guard device comprising: a support coupled to the portable MRI device; and a plurality of arms that, when the deployable guard device is in a deployed position, at least partially surround a region within which a magnetic field strength of a magnetic field generated by the portable MRI device equals or exceeds a threshold; and wherein applying a force to the first arm to move the first arm from its undeployed position toward its deployed position causes the support to move one or more other arms of the plurality of arms from their respective undeployed positions toward their respective deployed positions.

In some embodiments, the magnetic field strength of the magnetic field generated by the portable MRI device within the region is within a range from about 5 Gauss to about 70 Gauss. In some embodiments, the magnetic field generated by the portable MRI device within the region is within a range from about 1 Gauss to about 70 Gauss.

In some embodiments, the deployable guard device is coupled to the portable MRI device above an imaging region of the portable MRI device.

According to some aspects there is provided a method for operating a magnetic resonance imaging (MRI) device, the MRI device being coupled to a deployable guard device comprising a support coupled to the MRI device and a plurality of arms coupled to the support and that, when the deployable guard device is in a deployed position, at least partially surround a region within which a magnetic field strength of a magnetic field generated by the MRI device equals or exceeds a threshold, the method comprising: moving the deployable guard device from the deployed position to an undeployed position, wherein the deployable guard device is configured to move between the deployed position and the undeployed position in response to a force on only one of the plurality of arms; imaging, using the MRI device; and subsequent to imaging, moving the deployable guard device from the undeployed position to the deployed position.

The aspects and embodiments described above, as well as additional aspects and embodiments, are described further below. These aspects and/or embodiments may be used individually, all together, or in any combination, as the application is not limited in this respect.

For ease of explanation, embodiments of a deployable guard device disclosed herein are described in the context of a portable POC MRI device; however, it should be appreciated that such a guard device may also be used in conjunction with other devices including, but not limited to, X-ray images, CT imaging devices, etc.

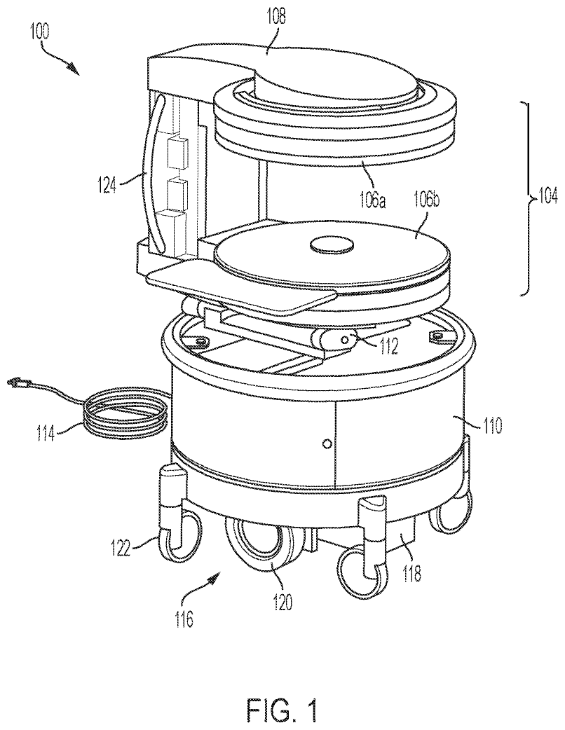

Referring initially to FIG. 1, there is shown an exemplary portable medical imaging device 100 for use in accordance with embodiments of the technology described herein. In the embodiment depicted in FIG. 1, the portable medical imaging device 100 may be a POC MRI device including a B₀ magnet 104 having at least one first permanent magnet 106a and at least one second permanent magnet 106b magnetically coupled to one another by a ferromagnetic yoke 108 configured to capture and channel magnetic flux to increase the magnetic flux density within the imaging region (field of view) of the MRI device 100. Alternatively, in some embodiments, B₀ magnet 104 may be formed using electromagnets, laminate magnets, or hybrid magnets. Additional information regarding the formation of B₀ magnet 104 may be found in U.S. Patent Publication No. US 2018/0143274, filed Nov. 22, 2017 and titled “Low-Field Magnetic Resonance Imaging Methods and Apparatus”, hereby incorporated by reference.

The medical imaging device 100 may comprise a low-field MRI device. As used herein, “low-field” refers generally to MRI devices operating with a B₀ field of less than or equal to approximately 0.2T, though systems having a B₀ field of between 0.2T and approximately 0.3T have sometimes been characterized as low-field as a consequence of increased field strengths at the high end of the high-field regime. Within the low-field regime, low-field MRI devices operating with a B₀ field of less than 0.IT are referred to herein as “very low-field” and low-field MRI devices operating with a B₀ field of less than 10 milliTesla (mT) are referred to herein as “ultra-low field”.

In some embodiments, the B₀ magnet 104 may be coupled to or otherwise attached or mounted to a base 110 by a positioning mechanism 112 (such as for example a goniometric stage) so that the B₀ magnet can be tilted (e.g., rotated about its center of mass) to provide an incline to accommodate a patient's anatomy as needed. In addition to providing a load bearing structure(s) for supporting the B₀ magnet 104, the base 110 may also include an interior space or compartment(s) configured to house the electronics (not shown) used to operate the portable MRI device 100. For example, the base 110 may house power components to operate gradient coils (e.g., X, Y and Z) and RF transmit/receive coils, as well as RF coil amplifiers (power amplifiers to operate the transmit/receive coils of the system), power supplies, console, power distribution unit and other electronics needed to operate the MRI device.

In some embodiments, the electronics needed to operate portable MRI device 100 may consume less than 1 kW of power and, in some embodiments, less than 750 W of power (e.g., MRI devices utilizing a permanent B₀ magnet solution). However, systems that consume greater power may also be utilized as well, as the aspects of the technology described herein are not limited in this respect. As such, the exemplary portable MRI device 100 may be powered via a single power connection 114 configured to connect to a source of mains electricity, such as an outlet providing single-phase power (e.g., a standard or large appliance outlet). Accordingly, the portable MRI device 100 can be plugged into a single available power outlet and operated therefrom. Aspects of power systems that may be used as part of portable MRI device 100 are described in U.S. Patent Publication No. US 2018/0143274, filed Nov. 22, 2017 and titled “Low-Field Magnetic Resonance Imaging Methods and Apparatus”, which is incorporated by reference in its entirety.

As further illustrated in FIG. 1, the portable MRI device 100 may also include a conveyance mechanism 116 that allows the portable MRI device 100 to be transported to different locations. The conveyance mechanism 116 may include one or more components configured to facilitate movement of the portable MRI device 100, for example, to a location at which MRI is needed. According to some embodiments, conveyance mechanism 116 may include a motor 118 coupled to drive wheels 120. In this manner, the conveyance mechanism 116 provides motorized assistance in transporting the MRI device 100 to desired locations. Additionally, the conveyance mechanism 116 may also include a plurality of casters 122 to assist with support and stability as well as facilitating transport.

In some embodiments, the conveyance mechanism 116 may optionally include motorized assistance controlled via a joystick (not shown) to guide the portable MRI device 100 during transportation to desired locations. According to some embodiments, the conveyance mechanism 116 may also include a power assist mechanism configured to detect when force is applied to the MRI device and, in response, to engage the conveyance mechanism 116 to provide motorized assistance in the direction of the detected force. For example, handles 124 may be configured to detect when force is applied thereto the rail (e.g., by personnel pushing on the handles 124) and engage the conveyance mechanism 116 to provide motorized assistance to drive the wheels 120 in the direction of the applied force. As a result, a user can guide the portable MRI device 100 with the assistance of the conveyance mechanism 116 that responds to the direction of force applied by the user.

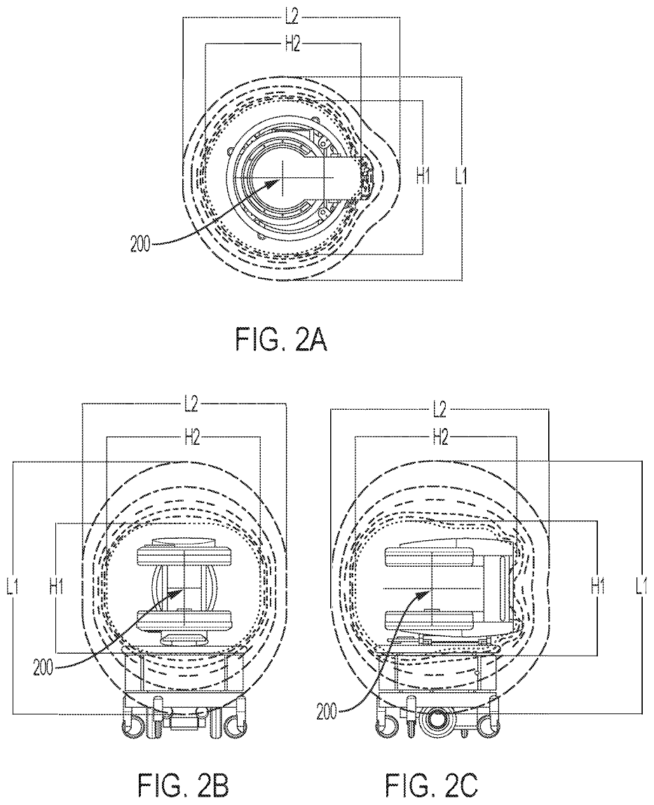

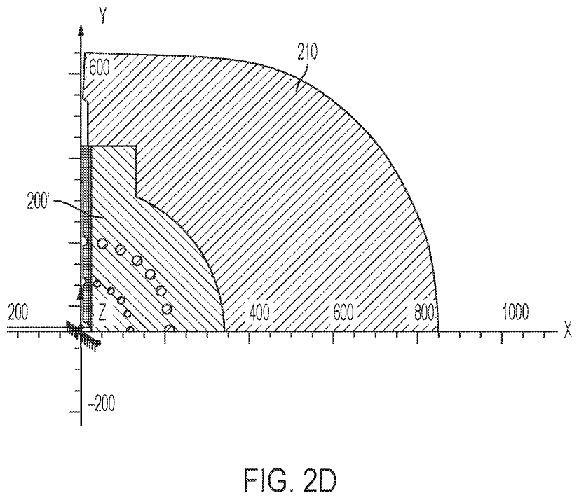

As indicated above, although the portable MRI device 100 operates at a B₀ field strength well below that of a traditional high-field system, there still may be concerns with access control, given certain fringe field strengths around an isocenter 200 of the B₀ magnet 104. By way of illustration, FIGS. 2A, 2B and 2C are top, front and side views, respectively of a portable medical imaging device, for example, the device shown in FIG. 1. For example, an innermost region (defined by dimensions H1 and H2) may represent a 30 Gauss region and an outermost region (defined by dimensions L1 and L2) may represent a 5 Gauss region, wherein the fringe field strength decreases with increasing distance from the isocenter 200. Thus, one consideration in this regard may be, for example, the International Electrotechnical Commission (IEC) 60601-2-33 standard, which defines controlled access as an area to which access is controlled for safety reasons. The standard further specifies that a controlled access area around the MR equipment shall be defined such that outside this area: 1) the magnetic fringe field strength shall not exceed 0.5 mT and 2) the electromagnetic interference level complies with IEC 60601-1-2.

FIG. 2D illustrates magnetic field strength of a fringe field for another example magnetic resonance imaging device, in accordance with some embodiments of the technology described herein. In some embodiments, the portable medical imaging device may generate a magnetic field which is asymmetrical. For example, as shown in FIG. 2D, the magnetic field 210 generated by MRI device 200′ is asymmetrical, extending farther along the x-axis than the y-axis. Accordingly, the fringe field generated by the MRI device 200′ which extends beyond the imaging region of the MRI device 200′ is also asymmetrical.

The inventors have developed a deployable guard device configured to move between an undeployed position and a deployed position. When in the deployed position, the deployable guard device may at least partially surround a region within which a magnetic field strength of a magnetic field generated by the portable MRI device equals or exceeds a threshold field strength. In some embodiments, the deployable guard device may be designed for use with an MRI device, such as MRI device 200′, which generates an asymmetric magnetic field. For example, the deployable guard device may be shaped to inhibit encroachment upon the high-strength asymmetric fringe fields. In some embodiments, the deployable guard device may comprise features which make transitioning the deployable guard device between the undeployed position and the deployed position easier.

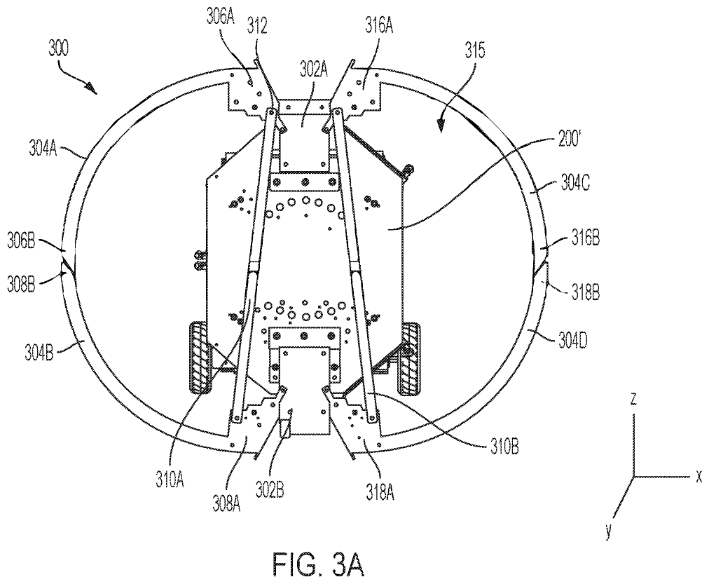

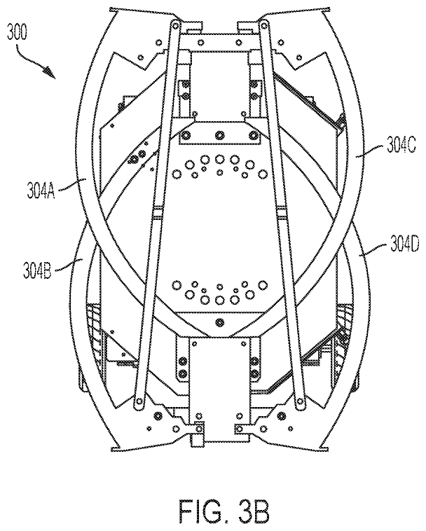

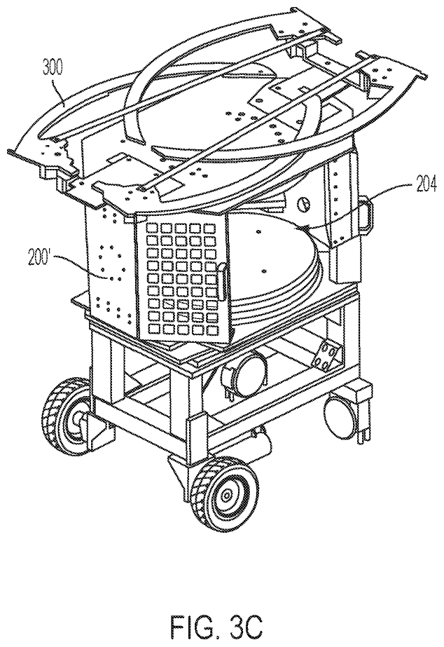

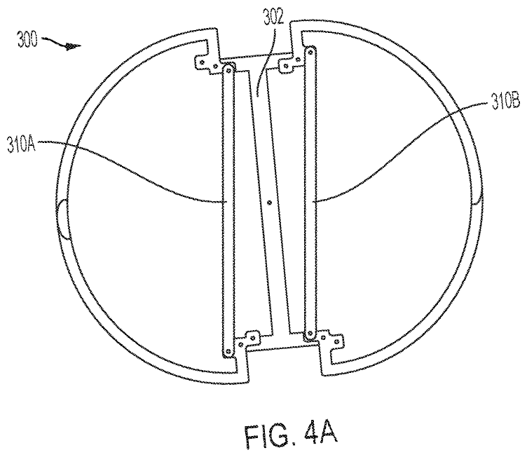

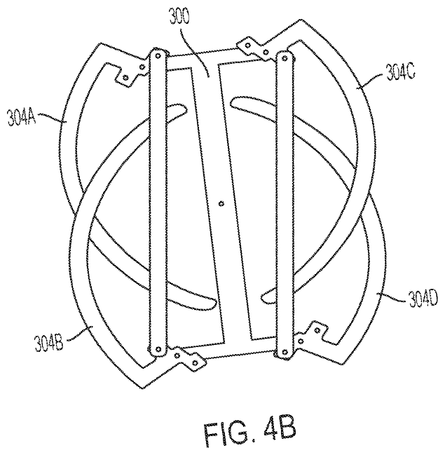

FIG. 3A illustrates an example deployable guard device 300 in a deployed position, in accordance with some embodiments of the technology described herein. As shown in FIG. 3A, the deployable guard device 300 comprises a plurality of arms 304A-D coupled to a base 302A-B.

The plurality of arms of the deployable guard device 300, when in a deployed position, at least partially surround a region 315 within which a magnetic field strength of a magnetic field generated by the portable MRI device to which the deployable guard device is coupled, equals or exceeds a threshold field strength. Accordingly, the plurality of arms may form a physical barrier which inhibits encroachment into the region 315. In some embodiments, the magnetic field strength of the magnetic field generated by the MRI device to which the deployable guard device 300 is coupled within the region 315 is within a range from about 5 Gauss to about 70 Gauss. In some embodiments, the magnetic field strength of the magnetic field generated by the MRI device to which the deployable guard device 300 is coupled within the region 315 is within a range from about 1 Gauss to about 70 Gauss.

The plurality of arms may comprise any suitable number of arms. For example, in the illustrated embodiment, the deployable guard device 300 comprises four arms 304A-D. The deployable guard device 300 may comprise two pairs of two arms, with one pair of arms on each side of the deployable guard device 300. As shown in FIG. 3A, the deployable guard device comprises first and second arms 304A-B on a first side of the deployable guard device, and third and fourth arms 304C-D on a second side of the deployable guard device.

The plurality of arms of the deployable guard device 300 may comprise any suitable material. In some embodiments, the plurality of arms comprise plastic. In some embodiments, the plurality of arms comprise G-10. The deployable guard device 300 may have any suitable size. In some embodiments, the deployable guard device may be sized such that the deployable guard device is able to be moved through a doorway when the deployable guard device is in the undeployed position. Accordingly, the deployable guard device may have a diameter of 32 inches or less in the undeployed position.

As described herein, the deployable guard device may be configured to inhibit encroachment upon an MRI device. In some embodiments, the deployable guard device 300, or portions thereof (e.g., at least some of the plurality of arms) may be colored with a bright color to maximize visibility and draw attention to the deployable guard device. In some embodiments, at least a portion of the deployable guard device (e.g., at least some of the plurality of arms, including a first arm) has a value, according to the Munsell color system, of at least 3, at least 4, at least 5, at least 6, at least 7, at least 8, at least 9, or 10.

Each of the arms 304A-D may be coupled to a base 302A-B of the deployable guard device 300. In the illustrated embodiment of FIG. 3A, the base comprises two portions 302A-B. In other embodiments, the base may comprise a single component. The portions of the base 302A-B are coupled together by connecting beams 310A-B.

Each arm 304A-D may be coupled to one of the portions of the base 302A-B at a first end of each respective arm. For example, first arm 304A is coupled to first base portion 302A at a first end 306A of the first arm 304A, second arm 304B is coupled to second base portion 302B at a first end 308B of the second arm 304B, third arm 304C is coupled to first base portion 302A at a first end 316A of the third arm 304C, and fourth arm 304D is coupled to second base portion 302B at a first end 318A of the fourth arm 304D. The first ends of the plurality of arms may be configured to pivot at a pivot point about the respective base portion to which the first end is coupled, allowing the plurality of arms 304A-D to move between undeployed and deployed positions. A representative pivot point 312 is shown in the illustrated embodiment of FIG. 3A for first arm 304A.

Each of the plurality of arms 304A-D further comprise second ends opposite from first ends. For example, first arm 304A comprises a second end 306B opposite first end 306A, second arm 304B comprises a second end 308B opposite first end 308A, third arm 304C comprises a second end 316B opposite first end 316A, and fourth arm 304D comprises a second end 318B opposite first end 318A. Pivoting of the first ends of the plurality of arms 304A-D causes second ends of the plurality of arms 304A-D to move towards or away from a center of the deployable guard device 300.

As shown in the illustrated embodiment of FIG. 3A, respective arms of the plurality of arms 304A-D may at least partially overlap each other when the deployable guard device 300 is in the deployed position. For example, as shown in FIG. 3A, first and second arms 304A-B at least partially overlap each other while third and fourth arms 304C-D at least partially overlap each other. More specifically, second end 306B of first arm 304A overlaps second end 308B of second arm 304B and second end 316B of third arm 304C overlaps second end 318B of fourth arm 318B. In some embodiments, the second end 308B of second arm 318B may overlap second end 306B of first arm 304A. In some embodiments, the second end 318B of fourth arm 304D may overlap second end 316B of third arm 304C.

The overlap of the plurality of arms may be along an axis which is perpendicular to a plane in which the region 315 at least partially surrounded by the deployable guard device 300 when in the deployed position lies. For example, in the illustrated embodiment of FIG. 3A, the deployable guard device 300 surrounds a region 315 which lies in the x-z plane. The overlap of respective ones of the plurality of arms 304A-D is along the y-axis (e.g., the vertical axis).

The respective ones of the plurality of arms 304A-D may overlap each other without being coupled together at the point of overlap. For example, second ends 306B, 308B of the first and second arms 304A-B overlap but are not coupled together. Second ends 316B, 318B of the third and fourth arms 304C-D overlap but are not coupled together.

In some embodiments, one or more of the plurality of arms may be configured to move in response to force on another one of the plurality of arms. For example, at least two arms of the plurality of arms (e.g., first arm 304A and second arm 304B, third arm 304C and fourth arm 304D) may be configured to move between the undeployed position and the deployed position in response to a force on only one of the at least two of the plurality of arms. Accordingly, the deployable guard device 300 may require less user interaction to transition the deployable guard device 300 between undeployed and deployed positions according to some aspects.

As described herein, the deployable guard device may be designed for inhibiting encroachment into a region with an asymmetrical magnetic field. Accordingly, the deployable guard device may be asymmetrical and thereby form a boundary which is asymmetrical. For example, the deployable guard device may be bilaterally asymmetrical. That is, the deployable guard device may lack symmetry about one or more perpendicular axes (e.g., the x- and z-axes). In the illustrated embodiment of FIG. 3A, the deployable guard device 300 is asymmetrical about the x- and z-axes. Likewise, the deployable guard device 300 lacks rotational symmetry.

In some embodiments, at least some of the plurality of arms 304A-D of the deployable guard device 300 are movable between the undeployed position and the deployed position without moving others of the plurality of arms 304A-D between the undeployed position and the deployed position. Accordingly, the deployable guard device 300 is able to be partially deployed. For example, one pair of the plurality of arms (e.g., first and second arms 304A-B) may be movable to the deployed position while a second pair of the plurality of arms (e.g., third and fourth arms 304C-D) remain in the undeployed position. The inventors have recognized that a partially deployable guard device may be advantageous, for example, in situations where the deployable guard device is in a confined space but deployment of at least a portion of the deployable guard device is still desired. For example, where the deployable guard device is stored in a corner of a room, the portion of the deployable guard device may remain in the undeployed position while the portion of the deployable guard device which is accessible may be in the deployed position.

FIGS. 3B-3C illustrate additional views of the deployable guard device 300. In particular, FIGS. 3B-3C illustrate the example deployable guard device 300 coupled to a portable magnetic resonance imaging device and in an undeployed position, in accordance with some embodiments of the technology described herein. As shown in FIGS. 3B-3C, the plurality of arms 304A-D pivot about pivot points at first ends of the plurality of arms towards a center of the deployable guard device 300 to move from the deployed position to the undeployed position. In the undeployed position, respective ones of the plurality of arms 304A-D overlap each other at points between first and second ends of the respective ones of the plurality of arms 304A-D.

FIG. 3C illustrates a perspective view of the deployable guard device 300 coupled to an MRI device 200′. As shown in the illustrated embodiment, the deployable guard device 300 is coupled to the MRI device 200′ above an imaging region 204 of the MRI device 200′. In other embodiments, the deployable guard device 300 may be coupled to the MRI device 200′ below the imaging region 204 of the MRI device 200′. In some embodiments, multiple deployable guard devices may be coupled to the MRI device 200′, for example, with one deployable guard device coupled below the imaging region 204 and one deployable guard device 300 coupled above the imaging region 204 of the MRI device 200′.

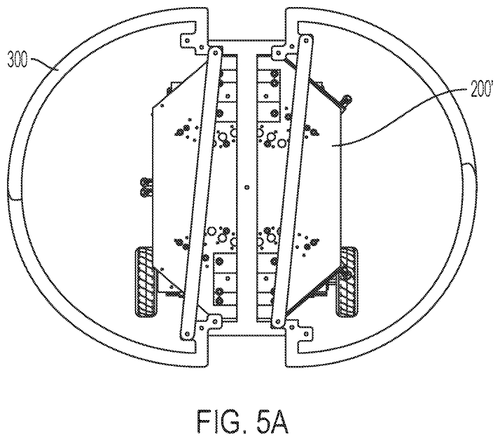

FIG. 4A illustrates another configuration of the deployable guard device 300 in a deployed position, in accordance with some embodiments of the technology described herein. In the illustrated embodiment of FIG. 4A, the base 302 of the deployable guard device 300 comprises a single portion. Further, in the illustrated embodiment of FIG. 4A, the connecting beams 310A-B are aligned parallel to each other. FIG. 4B illustrates the example deployable guard device of FIG. 4A in an undeployed position, in accordance with some embodiments of the technology described herein.

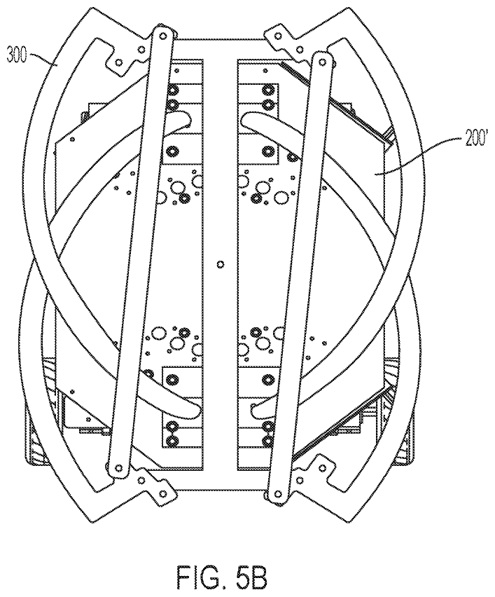

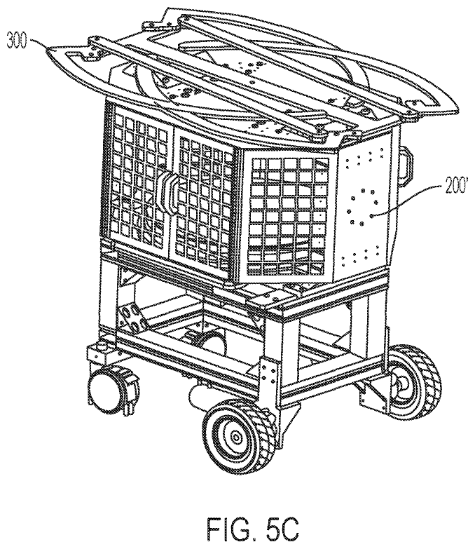

FIGS. 5A-5C illustrate the example deployable guard device of FIG. 4A coupled to a portable magnetic resonance imaging device, in accordance with some embodiments of the technology described herein. FIG. 5A illustrates the deployable guard device 300 in the undeployed position. FIGS. 5B-5C illustrate the deployable guard device 300 in the deployed position.

As described herein, in some embodiments, the deployable guard device 300 may be manually moved from an undeployed position to a deployed position and/or from a deployed position to an undeployed position. In some embodiments, the deployable guard device 300 may be configured to be automatically deployed (e.g., moved from an undeployed position to a deployed position) and/or retracted (e.g., moved from a deployed position to an undeployed position). For example, as described herein, the deployable guard device 300 may include a deployment device for automatically moving the deployable guard device between the undeployed and deployed positions.

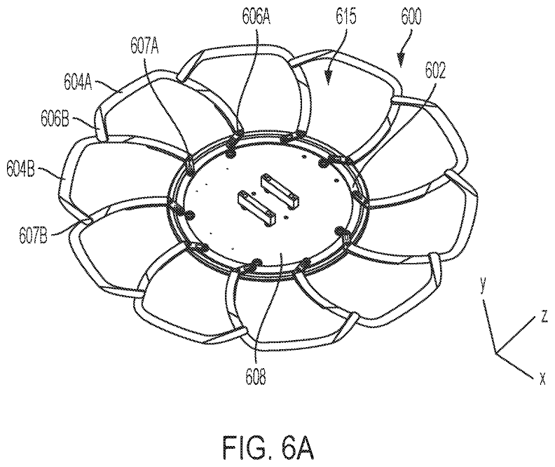

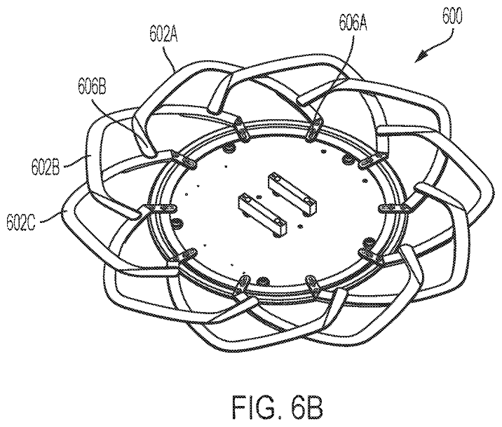

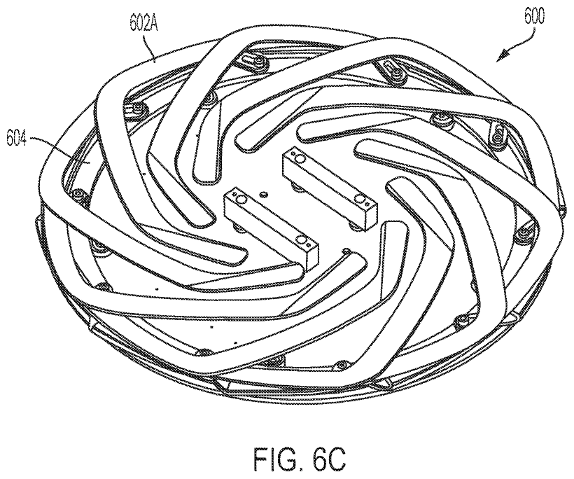

FIG. 6A illustrates another example deployable guard device 600, in accordance with some embodiments of the technology described herein. The deployable guard device 600 may be configured to transition between an undeployed position and a deployed position. In the deployed position, the deployable guard device 600 inhibits encroachment upon a portable medical imaging device. In the illustrated embodiment of FIG. 6A, the deployable guard device 600 is shown in the deployed position.

The deployable guard device 600 comprises a plurality of arms 604 coupled to a support 604. The plurality of arms 604 of the deployable guard device 600, when in a deployed position, at least partially surround a region 615 within which a magnetic field strength of a magnetic field generated by the portable MRI device to which the deployable guard device is coupled, equals or exceeds a threshold field strength. Accordingly, the plurality of arms 604 may form a physical barrier which inhibits encroachment into the region 615. In some embodiments, the magnetic field strength of the magnetic field generated by the MRI device to which the deployable guard device 600 is coupled within the region 615 is within a range from about 5 Gauss to about 70 Gauss. In some embodiments, the magnetic field strength of the magnetic field generated by the MRI device to which the deployable guard device 600 is coupled within the region 615 is within a range from about 1 Gauss to about 70 Gauss.

The plurality of arms may comprise any suitable number of arms. In some embodiments, the plurality of arms comprise at least 4 arms, at least 6 arms, at least 8 arms, at least 10 arms, or more. In the illustrated embodiment of FIG. 6A, the deployable guard device 600 comprises 10 arms, including, by way of example, first arm 604A and second arm 604B.

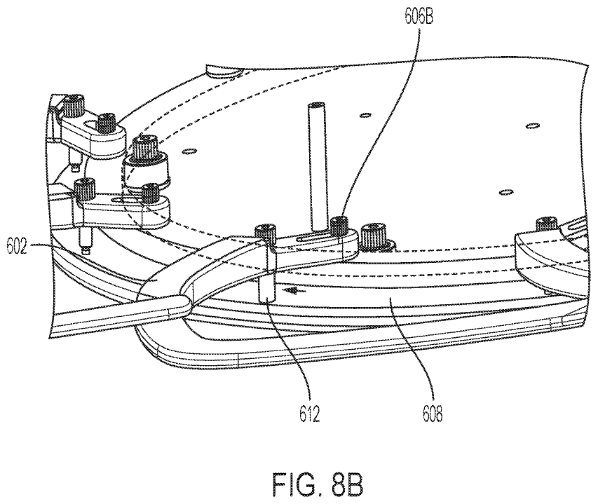

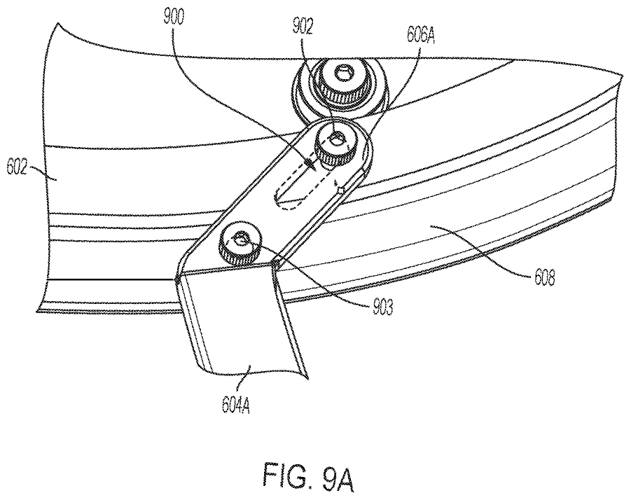

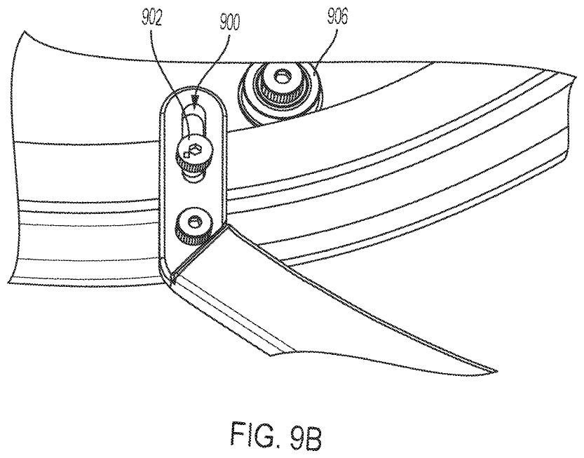

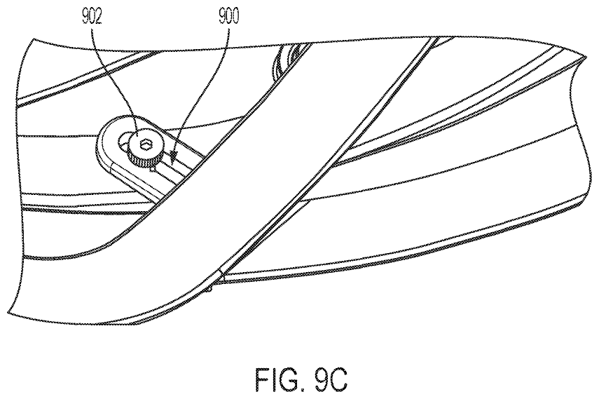

Each of the arms 604 may be coupled to a support 604. As described herein, the support may comprise a ring. The support may be rotatable, for example, when moving the deployable guard device 300 between the undeployed position and the deployed position. Further details of the rotation of the support 604 are described herein, for example, with reference to FIGS. 8A-9C.

The support 602 may be coupled to a base 608. In some embodiments, the base 608 comprises a track in which the support 602 is seated. The base 608 may be coupled to the portable MRI device, as is further shown and described herein.

Each arm 604 of the deployable guard device 600 may be coupled to the support 602 at a first end of each respective arm. For example, by way of example, first arm 604A is coupled to the support 602 at a first end 606A of the first arm 604A and second arm 604B is coupled to the support 602 at a first end 607A of the second arm 604B. The respective first ends of each arm 604 of the plurality of arms may slide along the support 602, allowing the plurality of arms to move between the undeployed position and the deployed position, as described herein.

Each of the plurality of arms further comprise second ends opposite from first ends. For example, first arm 604A comprises a second end 606B opposite first end 606A and second arm 604B comprises a second end 607B opposite first end 607A. As shown in the illustrated embodiment, each arm may comprise a fixed bend between first and second ends of the respective arm. Sliding of the first ends of the arms along the support 602 cause second ends of the plurality of arms 604 to move towards or away from a center of the deployable guard device 600.

The plurality of arms of the deployable guard device 600 may comprise any suitable material. In some embodiments, the plurality of arms comprise plastic. In some embodiments, the plurality of arms comprise G-10. The deployable guard device 600 may have any suitable size. In some embodiments, the deployable guard device may be sized such that the deployable guard device is able to be moved through a doorway when the deployable guard device is in the undeployed position. Accordingly, the deployable guard device may have a diameter of 32 inches or less in the undeployed position.

As described herein, the deployable guard device may be configured to inhibit encroachment upon an MRI device. In some embodiments, the deployable guard device 600, or portions thereof (e.g., at least some of the plurality of arms) may be colored with a bright color to maximize visibility and draw attention to the deployable guard device. In some embodiments, at least a portion of the deployable guard device (e.g., at least some of the plurality of arms, including a first arm) has a value, according to the Munsell color system, of at least 3, at least 4, at least 5, at least 6, at least 7, at least 8, at least 9, or 10.

As shown in the illustrated embodiment of FIG. 6A, respective arms of the plurality of arms may at least partially overlap each other when the deployable guard device 600 is in the deployed position. For example, as shown in FIG. 6A, first arm 604A at least partially overlaps second arm 604B. More specifically, second end 606B of first arm 604A overlaps second arm 604B at a point between first and second ends 607A-B of the second arm 604B.

The overlap of the plurality of arms may be along an axis which is perpendicular to a plane in which the region 615 at least partially surrounded by the deployable guard device 600 lies. For example, in the illustrated embodiment of FIG. 6A, the deployable guard device 600 surrounds a region 615 which lies in the x-z plane. The overlap of respective ones of the plurality of arms 604 is along the y-axis (e.g., the vertical axis).

The respective ones of the plurality of arms 604 may overlap others of the plurality of arms 604 without being coupled together at the point of overlap. For example, the second end 606B of the first arm 604A overlaps the second arm 606B without being coupled to the second arm 604B at the point between the first and second ends 607A-B of the second arm 604B which the second end 606B of the first arm 604A overlaps.

In some embodiments, one of more of the plurality of arms may be configured to move in response to a force on another one of the plurality of arms. For example, a force on a single arm (e.g., first arm 604A) of the plurality of arms may cause all of the arms of the plurality of arms to move between the undeployed position and the deployed position. Accordingly, the deployable guard device 600 may require less user interaction to transition deployable guard device 600 between undeployed and deployed positions according to some aspects.

In some embodiments, the deployable guard device may be rotationally symmetrical. For example, as shown in the illustrated embodiment of FIG. 6A, the deployable guard device 600 has rotational symmetry about the y-axis.

FIGS. 6B-6C illustrate additional views of the deployable guard device 600. FIG. 6B illustrates the deployable guard device of FIG. 6A in a position between the deployed position and an undeployed position, in accordance with some embodiments of the technology described herein. FIG. 6C illustrates the deployable guard device of FIG. 6A in the undeployed position, in accordance with some embodiments of the technology described herein. As shown in the illustrated embodiments of FIGS. 6B-6C, when the deployable guard device 600 moves from deployed position to the undeployed position, first ends of the plurality of arms slide along the support 602 and second ends of the plurality of arms move towards a center of the deployable guard device.

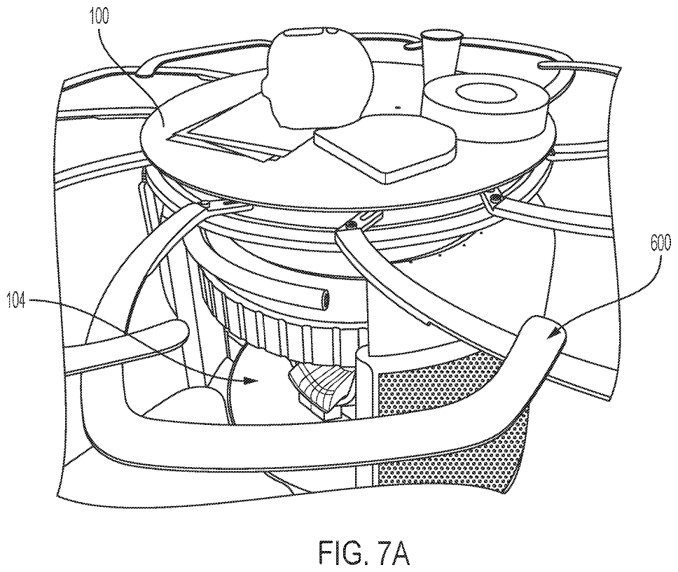

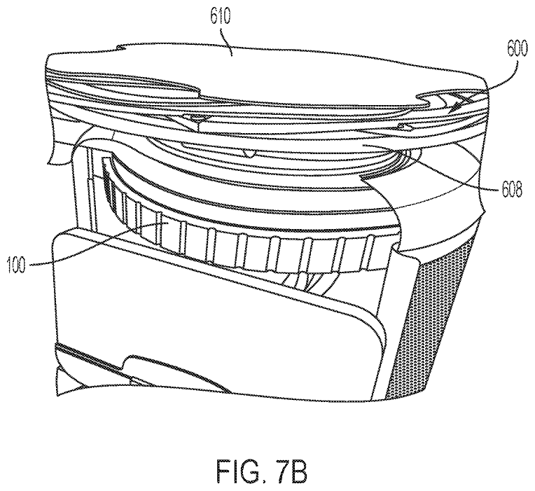

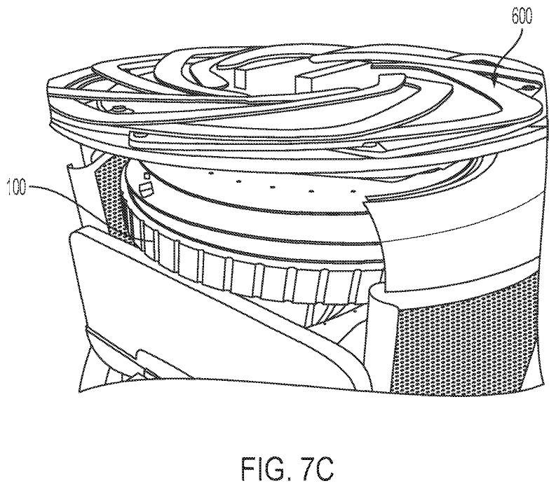

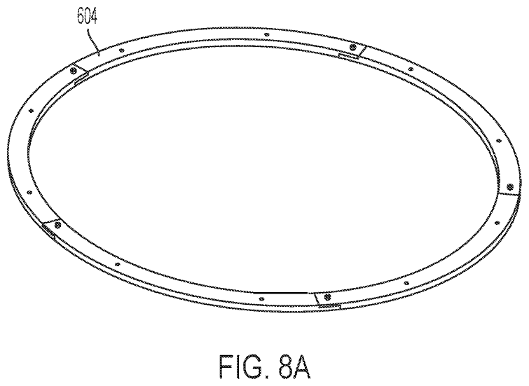

FIGS. 7A-7C illustrates the deployable guard device of FIG. 6A coupled to a portable magnetic resonance imaging device 100, in accordance with some embodiments of the technology described herein. FIG. 7A illustrates the deployable guard device 600 in the deployed position. FIGS. 7B-7C illustrate the deployable guard device in the undeployed position.

As shown in the illustrated embodiments of FIGS. 7A-7C, the deployable guard device 600 is coupled to the MRI device 100 above an imaging region 104 of the MRI device 100. In other embodiments, the deployable guard device 600 may be coupled to the MRI device 100 below the imaging region 104 of the MRI device 100. In some embodiments, multiple deployable guard devices may be coupled to the MRI device 100, for example, with one deployable guard device coupled below the imaging region 104 and one deployable guard device coupled above the imaging region 104 of the MRI device 100.

As shown in FIG. 7B, a base 608 of the deployable guard device 600 may be coupled to the MRI device 100. The base 608 may be disposed below the plurality of arms of the deployable guard device 600 and the support 602 of the deployable guard device. In some embodiments, the deployable guard device 600 further comprises a cover 610 disposed above the plurality of arms of the deployable guard device 600 and configured to at least partially house the plurality of arms of the deployable guard device 600 when the deployable guard device 600 is in the undeployed position.

FIGS. 8A-9C illustrate aspects of the support 602. FIG. 8A illustrates the support 602 of the deployable guard device 600. FIGS. 8B-9C illustrates aspects of coupling the deployable guard device 600 to the support 602, in accordance with some embodiments of the technology described herein.

As shown in FIGS. 8B-8C, the plurality of arms are coupled to the support 602. FIG. 9A illustrates first arm 604A being coupled to support 602 at first end 606A of the first arm 604A. The first end 606A of the first arm 604A comprises an opening 900 into which a first fastener 902 is inserted. First fastener 902 may comprise a screw, in some embodiments. The first fastener 902 is attached to support 602. The opening 900 is sized to allow for sliding of the first fastener 902 within the opening 900 (e.g., between endpoints of the opening 900). Accordingly, the first arm 904 is able to slide along the support 602.

The first arm 604A may, in some embodiments, be coupled to the base 608. As shown in the illustrated embodiments, the first arm 604A is coupled to the base 608 via second fastener 903. In some embodiments, second fastener 903 comprises a screw. Second fastener 903 may comprise a standoff 612 for spacing the first arm 604A from the base 608.

FIGS. 9A-9C illustrate motion of the deployable guard device of FIG. 6A when the deployable guard device is moved from the deployed position to the undeployed position. In particular, FIG. 9A illustrates components of the deployable guard device 600 in a deployed position. FIG. 9B illustrates components of the deployable guard device 600 in a position between the undeployed position and the deployed position. FIG. 9C illustrates components of the deployable guard device in the undeployed position.

When the deployable guard device 600 in the deployed position, the first fastener 902 is at an endpoint of the opening in the first arm 604A. As the deployable guard device 600 moves towards the deployed position, the first fastener 902 moves towards an opposite endpoint of the opening 900, as shown in FIG. 9B. As the first fastener 902 moves, the support 602 slides along a track of the base 608, and first arm 604 rotates about first fastener 902. In the undeployed position, as shown in FIG. 9C, the first arm 604 continues to rotate about first fastener 902 and first fastener 902 returns to its original position at the endpoint of the opening 900.

As described herein, the support 602 pivots along a track of base 608 when the first arm 604A is moved between the undeployed position and the deployed position. The deployable guard device 600 may comprise at least one bearing 906, which may be coupled to the base 906, to facilitate rotation of the support 602. Fasteners which couple others of the plurality of arms to the support 602 are caused to move due to rotation of the support 602. In turn, the others of the plurality of arms are able to transition between the undeployed position and the deployed position in response to a force on the first arm 604A alone.

Accordingly, in some embodiments, motion of one arm of the deployable guard device 600 affects motion of other arm(s) of the deployable guard device 600. In other embodiments, motion of at least some of the arms of the deployable guard device may be decoupled from each other. For example, in some embodiments, motion of one arm of the deployable guard device 600 may not cause a second arm of the deployable guard device to move. In some embodiments, at least some of the arms of the deployable guard device 600 are movable between the undeployed position and the deployed position without moving others of the plurality of arms between the undeployed position and the deployed position. Accordingly, the deployable guard device 600 is able to be partially deployed. For example, at least one arm of the arms of the deployable guard device 600 may be movable to the deployed position while at least one second arm remains in the undeployed position. As described herein, the inventors have recognized that a partially deployable guard device may be advantageous, for example, in situations where the deployable guard device is in a confined space but deployment of at least a portion of the deployable guard device is still desired. For example, where the deployable guard device is stored in a corner of a room, the portion of the deployable guard device may remain in the undeployed position while the portion of the deployable guard device which is accessible may be in the deployed position.

In some embodiments, transitioning between the undeployed position and the deployed position may be automated. For example, automated rotation of the support ring 602 may automatically cause the deployable guard device 600 to move between the undeployed position and the deployed position. Accordingly, in some embodiments, the deployable guard device 600 may be configured to be automatically deployed (e.g., moved from an undeployed position to a deployed position) and/or retracted (e.g., moved from a deployed position to an undeployed position). For example, as described herein, the deployable guard device 600 may include a deployment device for automatically moving the deployable guard device between the undeployed and deployed positions. In some embodiments, the deployable guard device 600 may be manually moved from an undeployed position to a deployed position and/or from a deployed position to an undeployed position.

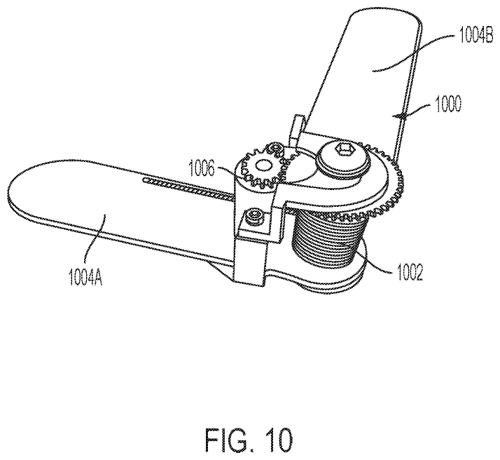

FIG. 10 illustrates a deployment device for automatically moving a deployable guard device from an undeployed position to a deployed position, in accordance with some embodiments of the technology described herein. As described herein, the deployable guard device, including the deployable guard devices 300, 600, may comprise features which facilitate transitioning the deployable guard device between the undeployed position and the deployed position. FIG. 10 illustrates an example deployment device 1000 configured to generate a force to facilitate moving the deployable guard device from an undeployed position to a deployed position.

In some embodiments, the deployment device 1000 may comprise a fixed portion 1004A coupled to a component of the deployable guard device which does not move when the deployable guard device transitions between the undeployed position and the deployed position (e.g., base 302, base 608). The deployment device 1000 may further comprise a movable portion 1004B coupled to at least one arm of the deployable guard device. The movable portion 1004B is configured to move towards and away from the fixed portion 1004A. That is, the deployment device 1000 may expand when the movable portion 1004B moves away from the fixed portion 1004A and contract when the movable portion 1004B moves towards the fixed portion 1004A. In the illustrated embodiment of FIG. 10, the deployment device 1000 is in an expanded position. In turn, the arms of the deployable guard device to which the movable portion 1004B is coupled are configured to move towards and away from the component to which the fixed portion 1004A is coupled automatically, via the deployment device.

The deployment device further comprises a deployment means configured to automatically return the deployment device 1000 to an expanded position. Accordingly, the deployment means is configured to automatically move the arms to which the movable portion 1004B is coupled away from the component of the deployable guard device to which the fixed portion 1004A is coupled. In turn, the deployment means comprises a means for automatically moving the deployable guard device from an undeployed position to a deployed position.

In some embodiments, the deployment means may be hydraulic. In some embodiments, the deployment means may be pneumatic. In some embodiments, the deployment means may be mechanical and/or electromechanical (e.g., including programmable control and one or more motors). In some embodiments, the deployment means may comprise a torsion spring 1002, in some embodiments. Force on the movable portion 1004B causes the torsion spring 1002 to turn inwards, building potential energy. When the force is removed, the built up potential energy causes the torsion spring 1002 to turn outwards, returning the movable portion 1004B to an expanded position. In some embodiments, the deployment means may further comprise a dampener 1006 for providing resistance against torsion spring 1002 to reduce the magnitude of the force which returns the movable portion 1004B to the expanded position.

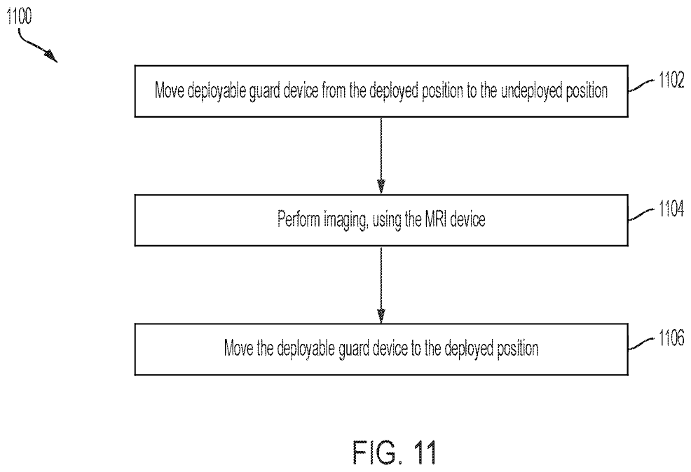

FIG. 11 illustrates a method for imaging using a magnetic resonance imaging device coupled to a deployable guard device, in accordance with some embodiments of the technology described herein. The method 1100 may begin at act 1102 where a deployable guard device coupled to an MRI device is moved from the deployed position to the undeployed position. In some embodiments, the deployable guard device comprises at least one deployment device configured to generate a first force to facilitate moving the deployable guard device from the undeployed position to the deployed position and moving the deployable guard device from the undeployed position to the deployed position comprises applying a second force on only one of the plurality of arms.

Optionally, the method 1100 may proceed to act 1104. At act 1104, imaging, using the MRI device may be performed. Subsequently, the method 1100 may proceed to act 1106. At act 1106, the deployable guard device may be moved from the undeployed position to the deployed position. In some embodiments, the MRI device may be transported to a second location subsequent to moving the deployable guard device to the deployed position.

The technology described herein may be embodied in any of the following configurations:
(1) A deployable guard device configured to be coupled to a portable magnetic resonance imaging (MRI) device, the deployable guard device comprising: a plurality of arms that, when the deployable guard device is in a deployed position, at least partially surround a region within which a magnetic field strength of a magnetic field generated by the portable MRI device equals or exceeds a threshold field strength; and at least one deployment device configured to generate a force to facilitate moving the deployable guard device from an undeployed position to the deployed position.
(2) The deployable guard device of (1), wherein the at least one deployment device comprises a torsion spring.
(3) The deployable guard device of (1) or (2), wherein the at least one deployment device comprises a respective deployment device coupled to each of the plurality of arms.
(4) The deployable guard device of any one of (1) through (3), wherein the deployable guard device is radially asymmetrical.
(5) The deployable guard device of any one of (1) through (4), wherein the deployable guard device is bilaterally asymmetrical.
(6) The deployable guard device of any one of (1) through (5), wherein at least some arms of the plurality of arms are movable to the deployed position while others of the plurality of arms remain in an undeployed position.
(7) The deployable guard device of any one of (1) through (6), wherein: the plurality of arms comprises a first arm and a second arm; and the first and second arms at least partially overlap one another in space when the deployable guard device is in the deployed position.
(8) The deployable guard device of (7), wherein the first and second arms are not coupled together at a point where the first and second arms at least partially overlap each other.
(9) The deployable guard device of (7), wherein a first end of the first arm overlaps a first end of the second arm when the deployable guard device is in the deployed position.
(10) The deployable guard device of any one of (1) through (9), wherein the plurality of arms comprises at least four arms.
(11) The deployable guard device of any one of (1) through (10), wherein at least two arms of the plurality of arms are configured to move from the deployed position to the undeployed position in response to a force on only one of the at least two arms the plurality of arms.
(12) The deployable guard device of any one of (1) through (11), wherein at least two arms of the plurality of arms are configured to move from the undeployed position to the deployed position in response to a force on only one of the at least two of the plurality of arms.
(13) The deployable guard device of (2), wherein the at least one deployment device is configured to automatically return the deployable guard device to the deployed position in response to a force on the deployable guard device towards the undeployed position.
(14) A deployable guard device configured to be coupled to a portable magnetic resonance imaging (MRI) device, the deployable guard device comprising: a plurality of arms that, when the deployable guard device is in a deployed position, at least partially surround a region within which a magnetic field strength of a magnetic field generated by the portable MRI device equals or exceeds a threshold field strength; and at least one deployment means for moving the deployable guard device from an undeployed position to the deployed position.
(15) The deployable guard device of (14), wherein the at least one deployment means comprises a respective deployment means for each of the plurality of arms.
(16) The deployable guard device of any one of (14) or (15), wherein the deployable guard device is radially asymmetrical.
(17) The deployable guard device of any one of (14) through (16), wherein the deployable guard device is bilaterally asymmetrical.
(18) The deployable guard device of any one of (14) through (17), wherein at least some arms of the plurality of arms are movable to the deployed position while others of the plurality of arms remain in an undeployed position.
(19) The deployable guard device of any one of (14) through (18), wherein the plurality of arms comprise a first arm and a second arm and the first and second arms at least partially overlaps one another in space when the deployable guard device is in the deployed position.
(20) The deployable guard device of (19), wherein the first and second arms are not coupled together at a point where the first and second arms at least partially overlap each other.
(21) The deployable guard device of (19), wherein a first end of the first arm overlaps a first end of the second arm when the deployable guard device is in the deployed position.
(22) The deployable guard device of any one of (14) through (21), wherein the plurality of arms comprise at least four arms.
(23) The deployable guard device of any one of (14) through (22), wherein at least two arms of the plurality of arms are configured to move from the deployed position to the undeployed position in response to a force on only one of the at least two arms the plurality of arms.
(24) The deployable guard device of any one of (14) through (23), wherein at least two arms of the plurality of arms are configured to move from the undeployed position to the deployed position in response to a force on only one of the at least two of the plurality of arms.
(25) A system, comprising: a portable magnetic resonance imaging (MRI) device; and a deployable guard device coupled to the portable MRI device, the deployable guard device comprising: a plurality of arms that, when the deployable guard device is in a deployed position, at least partially surround a region within which a magnetic field strength of a magnetic field generated by the portable MRI device equals or exceeds a threshold field strength; and at least one deployment device configured to generate a force to facilitate moving the deployable guard device from an undeployed position to the deployed position.
(26) The system of (25), wherein the magnetic field strength of the magnetic field generated by the portable MRI device within the region is within a range from about 5 Gauss to about 70 Gauss.
(27) The system of (25) or (26), wherein the magnetic field strength of the magnetic field generated by the portable MRI device within the region is within a range from about 1 Gauss to about 70 Gauss.
(28) The system of any one of (25) through (27), wherein the deployable guard device is coupled to the portable MRI device above an imaging region of the portable MRI device.
(29) A method for operating a magnetic resonance imaging (MRI) device, the MRI device being coupled to a deployable guard device comprising a plurality of arms that, when the deployable guard device is in a deployed position, at least partially surround a region within which a magnetic field strength of a magnetic field generated by the MRI device equals or exceeds a threshold, the method comprising: moving the deployable guard device from an undeployed position to the deployed position, wherein: the deployable guard device further comprises at least one deployment device configured to generate a first force to facilitate moving the deployable guard device from the undeployed position to the deployed position; subsequent to moving the deployable guard device to the deployed position, transporting the MRI device from a first location to a second location; and moving the deployable guard device from the deployed position to the undeployed position.
(30) The method of (29), wherein moving the deployable guard device from the undeployed position to the deployed position comprises applying a second force on only one of the plurality of arms.
(31) The method of (30), further comprising, subsequent to moving the deployable guard device to the undeployed position, imaging, using the MRI device.
(32) A deployable guard device configured to be coupled to a portable magnetic resonance imaging (MRI) device, the deployable guard device comprising: a support for coupling to the portable MRI device; and a plurality of arms coupled to the support and that, when the deployable guard device is in a deployed position, at least partially surround a region within which a magnetic field strength of a magnetic field generated by the portable MRI device equals or exceeds a threshold field strength, the plurality of arms including a first arm, wherein applying a force to the first arm to move the first arm from its undeployed position toward its deployed position causes the support to move one or more other arms of the plurality of arms from their respective undeployed positions toward their respective deployed positions.
(33) The deployable guard device of (32), wherein applying a second force to the first arm to move the first arm from its deployed position toward its undeployed position causes the support to move the one or more other arms of the plurality of arms from their respective deployed positions toward their respective undeployed positions.
(34) The deployable guard device of (32) or (33), wherein first ends of each of the plurality of arms slide along the support when the deployable guard device moves between the deployed position and the undeployed position.
(35) The deployable guard device of any one of (32) through (34), wherein the support is rotatable.
(36) The deployable guard device of (35), wherein the support rotates along a track coupled to the portable MRI device.
(37) The deployable guard device of any one of (32) through (36), wherein the first arm of the plurality of arms comprises plastic.
(38) The deployable guard device of any one of (32) through (37), wherein the first arm of the plurality of arms comprises a fixed bend between endpoints of the first arm.
(39) The deployable guard device of any one of (32) through (38), wherein the first arm of the plurality of arms has a color value of at least 3.
(40) The deployable guard device of any one of (32) through (39), wherein a second end of the first arm overlaps a second arm of the plurality of arms when the deployable guard device is in the deployed position.
(41) The deployable guard device of (35), wherein the second end of the first arm is not coupled to the second arm.
(42) The deployable guard device of (35), wherein the second end of the first arm overlaps a point on the second arm between first and second ends of the second arm when the deployable guard device is in the deployed position.
(43) The deployable guard device of any one of (32) through (42), wherein the plurality of arms comprises at least 8 arms.
(44) A system, comprising: a portable magnetic resonance imaging (MRI) device; and a deployable guard device coupled to the portable MRI device, the deployable guard device comprising: a support coupled to the portable MRI device; and a plurality of arms that, when the deployable guard device is in a deployed position, at least partially surround a region within which a magnetic field strength of a magnetic field generated by the portable MRI device equals or exceeds a threshold; and wherein applying a force to the first arm to move the first arm from its undeployed position toward its deployed position causes the support to move one or more other arms of the plurality of arms from their respective undeployed positions toward their respective deployed positions.
(45) The system of (44), wherein the magnetic field strength of the magnetic field generated by the portable MRI device within the region is within a range from about 5 Gauss to about 70 Gauss.
(46) The system of (44) or (45), wherein the magnetic field strength of the magnetic field generated by the portable MRI device within the region is within a range from about 1 Gauss to about 70 Gauss.
(47) The system of any one of (44) through (46), wherein the deployable guard device is coupled to the portable MRI device above an imaging region of the portable MRI device.
(48) A method for operating a magnetic resonance imaging (MRI) device, the MRI device being coupled to a deployable guard device comprising a support coupled to the MRI device and a plurality of arms coupled to the support and that, when the deployable guard device is in a deployed position, at least partially surround a region within which a magnetic field strength of a magnetic field generated by the MRI device equals or exceeds a threshold, the method comprising: moving the deployable guard device from the deployed position to an undeployed position, wherein the deployable guard device is configured to move between the deployed position and the undeployed position in response to a force on only one of the plurality of arms; imaging, using the MRI device; and subsequent to imaging, moving the deployable guard device from the undeployed position to the deployed position.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately”, “substantially,” and “about” may be used to mean within +20% of a target value in some embodiments, within +10% of a target value in some embodiments, within +5% of a target value in some embodiments, and within +2% of a target value in some embodiments. The terms “approximately” and “about” may include the target value.

What is claimed is:

1. A deployable guard device configured to be coupled to a portable magnetic resonance imaging (MRI) device, the deployable guard device comprising:
a plurality of arms that, when the deployable guard device is in a deployed position, at least partially surround a region within which a magnetic field strength of a magnetic field generated by the portable MRI device equals or exceeds a threshold field strength; and
at least one deployment device configured to generate a force to facilitate moving the deployable guard device from an undeployed position to the deployed position.

2. The deployable guard device of claim 1, wherein the at least one deployment device comprises a torsion spring.

3. The deployable guard device of claim 1, wherein the at least one deployment device comprises a respective deployment device coupled to each of the plurality of arms.

4. The deployable guard device of claim 1, wherein the deployable guard device is radially asymmetrical.

5. The deployable guard device of claim 1, wherein the deployable guard device is bilaterally asymmetrical.

6. The deployable guard device of claim 1, wherein at least some arms of the plurality of arms are movable to the deployed position while others of the plurality of arms remain in an undeployed position.

7. The deployable guard device of claim 1, wherein:
the plurality of arms comprises a first arm and a second arm; and
the first and second arms at least partially overlap one another in space when the deployable guard device is in the deployed position.

8. The deployable guard device of claim 7, wherein the first and second arms are not coupled together at a point where the first and second arms at least partially overlap each other.

9. The deployable guard device of claim 7, wherein a first end of the first arm overlaps a first end of the second arm when the deployable guard device is in the deployed position.

10. The deployable guard device of claim 1, wherein the plurality of arms comprises at least four arms.

11. The deployable guard device of claim 1, wherein at least two arms of the plurality of arms are configured to move from the deployed position to the undeployed position in response to a force on only one of the at least two arms the plurality of arms.

12. The deployable guard device of claim 1, wherein at least two arms of the plurality of arms are configured to move from the undeployed position to the deployed position in response to a force on only one of the at least two of the plurality of arms.

13. The deployable guard device of claim 1, wherein the at least one deployment device is configured to automatically return the deployable guard device to the deployed position in response to a force on the deployable guard device towards the undeployed position.

14. A system, comprising:
a portable magnetic resonance imaging (MRI) device; and
a deployable guard device coupled to the portable MRI device, the deployable guard device comprising:
a plurality of arms that, when the deployable guard device is in a deployed position, at least partially surround a region within which a magnetic field strength of a magnetic field generated by the portable MRI device equals or exceeds a threshold field strength; and
at least one deployment device configured to generate a force to facilitate moving the deployable guard device from an undeployed position to the deployed position.

15. A method for operating a magnetic resonance imaging (MRI) device, the MRI device being coupled to a deployable guard device comprising a plurality of arms that, when the deployable guard device is in a deployed position, at least partially surround a region within which a magnetic field strength of a magnetic field generated by the MRI device equals or exceeds a threshold, the method comprising:
moving the deployable guard device from an undeployed position to the deployed position, wherein:
the deployable guard device further comprises at least one deployment device configured to generate a first force to facilitate moving the deployable guard device from the undeployed position to the deployed position;
subsequent to moving the deployable guard device to the deployed position, transporting the MRI device from a first location to a second location; and
moving the deployable guard device from the deployed position to the undeployed position.
